(19) United States
(12) Patent Application Publication
(10) Pub. No.: US 2025/0108658 A1
(43) Pub. Date: Apr. 3, 2025

(54) INKJET PRINTING PROCESSES FOR TEMPORARY TATTOOS

(71) Applicant: Prism Inks, Inc., Sunnyvale, CA (US)

(72) Inventors: Amir A. AJANEE, Saratoga, CA (US); Graham A. DRACUP, Sunnyvale, CA (US); Carlos J. HERNANDEZ, San Jose, CA (US); Omar K. AJANEE, Saratoga, CA (US); Travis T. CALHOUN, San Jose, CA (US)

(73) Assignee: Prism Inks, Inc., Sunnyvale, CA (US)

(21) Appl. No.: 18/897,698

(22) Filed: Sep. 26, 2024

Related U.S. Application Data
(60) Provisional application No. 63/586,687, filed on Sep. 29, 2023.

(51) Int. Cl. B44C 1/16; A61K 8/02; A61Q 1/02; B41M 3/12

(52) U.S. Cl. CPC B44C 1/16; A61K 8/02; A61Q 1/02; B41M 3/12

(57) ABSTRACT

Provided are temporary tattoos comprising cosmetic inkjet ink and methods of making thereof. The method of making the temporary tattoo may comprise printing, using an inkjet printer, an image made from a cosmetic inkjet ink on a side of a transfer film that comprises an inkjet receptive coating. The image printed on the transfer film may be dried to allow the image to absorb into the coating. Once dried, an adhesive may be applied to the side of the transfer film comprising the inkjet receptive coating to cover the image. Thus, the temporary tattoo may comprise a transfer film comprising an inkjet receptive coating, and an adhesive film.

### CROSS-REFERENCE TO RELATED APPLICATIONS

This application claims priority to and the benefit of U.S. Provisional Application No. 63/586,687, filed Sep. 29, 2023, the contents of which are incorporated herein by reference in their entirety.

### FIELD

This disclosure relates to temporary tattoos, and more specifically, to producing temporary tattoos using inkjet printing processes with cosmetic inkjet ink.

### BACKGROUND

Temporary tattoos are typically printed using a screen-printing process. Screen printing requires a stencil to be produced using a printed image that has UV light exposed through it to a light sensitive emulsion. The inverse image that is exposed on the mesh is then used to apply the color to the film to produce the temporary tattoo. The time and money in the creation and use of the stencils for screen printing requires a company to do long print runs of the film to ensure they obtain a positive return on their investment.

### SUMMARY

Provided herein are temporary tattoos produced using cosmetic inkjet ink and methods of making temporary tattoos using cosmetic inkjet printing processes. The methods can include using an inkjet printer to print an image made of food, drug, cosmetic grade, or other colorant inkjet ink on a specialized transfer film comprising an inkjet receptive coating. The method may also include drying the image on the transfer film, for example by air-drying or applying heat to the transfer film to allow the ink to absorb into the coating. Finally, the method can include an adhesive film to the transfer film to cover the image, thereby producing a temporary tattoo that can then be packaged and/or used by a consumer.

The temporary tattoos made using cosmetic inkjet ink as described herein may exhibit various benefits over existing temporary tattoo products. The temporary tattoos described herein can be produced using a cosmetic-grade ink that is compatible with inkjet printers to generate images and designs for temporary tattoos with high precision and quality. Using inkjet printing processes, small batches of temporary tattoos can easily be produced, thereby removing barriers to entry in producing small amounts of temporary tattoos at a low cost and with minimal product waste. Further, the cosmetic inkjet ink composition in combination with the specialized transfer film composition described herein may provide for a temporary tattoo that, once applied, has a greater resolution, and lasts significantly longer than conventional temporary tattoos. The coloring of the temporary tattoos described herein may also improve upon existing temporary tattoos because the cosmetic inkjet ink described herein may utilize organic pigments and/or dyes that are more vibrant than, for example, conventional dye lakes and inorganic pigments. Also, the temporary tattoos described herein may have improved washability and abrasion-resistance. The temporary tattoos made using the cosmetic inkjet ink described herein may be easily removed when desired by the user using only water and soap or by directly peeling the tattoo off the skin.

In some examples, a method of making a temporary tattoo is provided, comprising: printing, using an inkjet printer, an image made from a cosmetic inkjet ink on a side of a transfer film comprising an inkjet receptive coating; drying the image printed on the transfer film such that the image absorbs into the inkjet receptive coating; and applying an adhesive film to the side of the transfer film comprising the inkjet receptive coating to cover the image. In some examples, the printed image has a resolution of 360-2440 vertical or horizontal dots per inch (dpi). In some examples, the inkjet receptive coating has a thickness between 1-10 microns. In some examples, the transfer film comprises a print backing, a water-soluble layer on a side of the print backing, and a protective polymer layer on a side of the water-soluble layer opposite the print backing. In some examples, the inkjet receptive coating is on a side of the protective polymer layer opposite the water-soluble layer. In some examples, the protective polymer layer has a thickness of 1-10 microns. In some examples, the adhesive film comprises a backing, a wax layer on a side of the backing, and an adhesive layer on a side of the wax layer opposite the backing. In some examples, applying the adhesive film to the side of the transfer film comprising the inkjet receptive coating comprises applying the adhesive layer of the adhesive film to the inkjet receptive coating of the transfer film. In some examples, the inkjet printer comprises piezo printheads or thermal printheads. In some examples, drying the image comprises air-drying or using heat to dry the image on the transfer film.

In some examples, a temporary tattoo having a cosmetic inkjet ink-printed image is provided, comprising: a print backing; a water-soluble layer on a side of the print backing; a protective polymer layer on a side of the water-soluble layer opposite the print backing; a cosmetic ink layer on a side of the protective polymer layer opposite the water-soluble film, the cosmetic ink layer comprising: an inkjet receptive coating; and a cosmetic inkjet ink absorbed into the inkjet receptive coating; and an adhesive layer on a side of the cosmetic ink layer opposite the protective polymer film. In some examples, the cosmetic ink layer has a resolution of 360-2440 horizontal or vertical dpi. In some examples, the inkjet receptive coating has a thickness of 1-10 microns. In some examples, the protective polymer layer has a thickness of 1-10 microns. In some examples, the cosmetic inkjet ink comprises 10-25 wt. % humectant. In some examples, the humectant comprises one or more of diglycerol, glycerin isoprene glycol, dipropylene glycol, tripropylene glycol monomethyl ether, and triethylene glycol monobutyl ether. In some examples, the cosmetic inkjet ink comprises 1-15 wt. % polymer. In some examples, the polymer comprises polyethylene glycol or polyvinylpyrrolidone. In some examples, the cosmetic inkjet ink comprises 0.01-2 wt. % surfactant. In some examples, the cosmetic inkjet ink comprises 2-20 wt. % of one or more cosmetic approved dyes and/or cosmetic approved pigments. In some examples, the cosmetic inkjet ink comprises 0.01-2 wt. % pH buffer. In some examples, the pH buffer comprises dimethylaminopropylamine or aminomethyl propanediol. In some examples, the cosmetic inkjet ink comprises 0.1-3 wt. % biocide. In some examples, the biocide comprises one or more of benzothiazolinone, phenoxyethanol, and ethylhexylglycerin. In some examples, the cosmetic inkjet ink comprises 60-80 wt. % solvent. In some examples, the solvent comprises water or ethanol. In some examples, the print backing is a porous paper-based substrate. In some examples, the protective polymer layer comprises acrylic and/or polyurethane. In some examples, the water-soluble layer comprises polyvinyl alcohol. In some examples, the temporary tattoo comprises an adhesive film, the adhesive film comprising a backing, a wax layer on a side of the backing, and the adhesive layer on a side of the wax layer opposite the backing. In some examples, the backing comprises polyethylene terephthalate.

### BRIEF DESCRIPTION OF THE FIGURES

FIG. 1 shows a cross-sectional view of a temporary tattoo produced using cosmetic inkjet ink, according to some aspects.

FIG. 2A shows a cross-sectional view of cosmetic ink being printed on a transfer film using inkjet printing processes, according to some aspects.

FIG. 2B shows a cross-sectional view of an image made from the cosmetic inkjet ink drying on the transfer film, according to some aspects.

FIG. 2C shows a cross-sectional view of the adhesive film being applied to the transfer film to form the temporary tattoo, according to some aspects.

FIG. 3A shows a cross-sectional view of a temporary tattoo being applied to skin by applying water to a print backing, according to some aspects.

FIG. 3B shows a cross-sectional view of the wet print backing of the temporary tattoo being removed, according to some aspects.

FIG. 3C shows a cross-sectional view of the final temporary tattoo applied to the skin, according to some aspects.

FIGS. 4A, 4B, 4C, and 4D show various cosmetic inkjet ink images printed on an inkjet receptive coating of a transfer film, according to some aspects.

FIG. 5 shows the petri dish setup for testing re-solubility of polymer/dye solutions, according to some aspects.

### DETAILED DESCRIPTION

Provided are methods of temporary tattoo production using cosmetic inkjet ink and temporary tattoos made using the methods and processes described herein. The temporary tattoos described herein may be produced by printing an image made from cosmetic inkjet ink on an inkjet receptive coating of a transfer film with an inkjet printer. The image can be dried using heat and/or by air-drying the tattoo to allow the ink to absorb into the coating. Once dried, an adhesive film can be applied to the transfer film to cover the image, thereby producing a temporary tattoo that is ready to be used.

The methods described herein may improve upon existing temporary tattoo production methods because the described methods utilize inkjet printing processes, which have heretofore not been usable in producing temporary tattoos, at least because temporary tattoos require cosmetic-grade ink that is not compatible with inkjet printing processes. The methods and temporary tattoos described herein harness the benefits of inkjet printing by realizing a cosmetic ink formulation capable of being used with inkjet printing processes for producing temporary tattoos. Using the formulations and methods described herein, a temporary tattoo that is abrasion-resistant, long lasting, vibrant in color, and non-tacky can be provided. Moreover, the temporary tattoos described herein may exhibit improved washability and have a high degree of precision compared to conventional temporary tattoos producing using screen-printing processes.

Inkjet printing processes introduce several benefits. First, they allow for efficient, small-batch production of customizable tattoos at a low cost and with minimal waste. This is an improvement over existing temporary tattoo production methods, which typically utilize screen-printing (i.e., silk-printing) processes that necessitate production of large batches to see a profit and can result in product waste. Also, inkjet printing processes achieve a degree of precision that is unattainable by existing temporary tattoo production methods (e.g., screen-printing).

The term “temporary” is used herein to describe the tattoos produced using cosmetic inkjet ink and is otherwise understood to mean “semi-permanent.” As described herein, the temporary tattoos produced using the cosmetic inkjet ink may withstand abrasion and exhibit water-resistance unless soap and water is applied to the temporary tattoo on the skin. In this instance, the temporary tattoo may be easily washed off of the skin. In fact, the temporary tattoos described herein may demonstrate improved washability in comparison to conventional temporary tattoos.

Described in greater detail below are (1) temporary tattoos made using cosmetic inkjet ink; (2) cosmetic inkjet ink compositions; (3) methods of making temporary tattoos using cosmetic inkjet ink; and (4) methods of applying the temporary tattoos provided herein.

### Temporary Tattoos Made Using Cosmetic Inkjet Ink

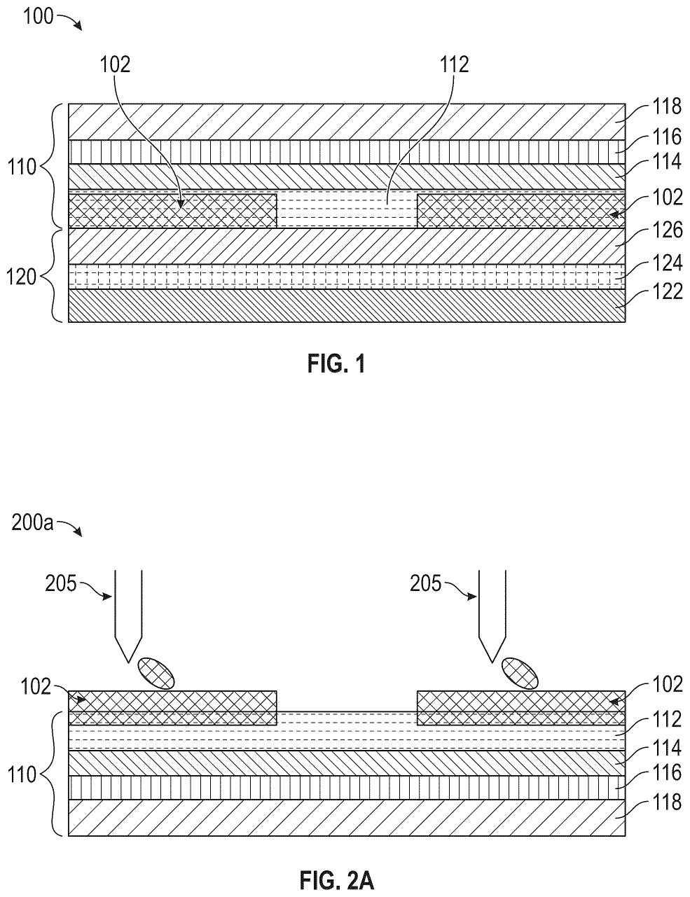

FIG. 1 illustrates a cross-sectional view of a temporary tattoo 100 comprising a cosmetic inkjet ink-printed image. The temporary tattoo 100 may comprise a transfer film 110 and an adhesive film 120, each of which will be described in greater detail below.

The transfer film 110 may comprise a print backing 118, a water-soluble layer 116 on a side of the print backing 118, a protective polymer layer 114 on a side of the water-soluble layer 116 opposite the print backing 118, and a cosmetic ink layer on a side of the protective polymer layer 114 opposite the water-soluble layer 116. The cosmetic ink layer may comprise an inkjet receptive coating 112 and a cosmetic inkjet ink 102 absorbed into the inkjet receptive coating 112. The order of the layers may be important to ensure the waterproof and adhesion characteristics of each of the layers are appropriately enabled. The efficacy of waterproofing and adhesion of the temporary tattoo may be based on the compositions of the layers of the temporary tattoo, examples of which are described herein.

The print backing 118 may comprise a porous paper-based substrate. The pores can allow water to migrate through the print backing 118 to the water-soluble layer 116, causing the print backing 118 to be easily removed. Example print backing 118 substrates may include cellulose- and/or synthetic-based substrates with sufficient porosity to allow water to penetrate through the substrate.

The water-soluble layer 116 may comprise polyvinyl alcohol, polyvinylpyrrolidone, polyacrylic acid, polyethylene glycols, cationic polyquaterniums, polyethylene oxide, polyvinylamine, polyacrylamide, polymethacrylic acid, poly (N,N-dimethyl acrylamide), polyethyl oxazoline, poly styrene sulfonic acid, polyvinyl pyridine, hydroxy ethyl cellulose, hydroxy propyl cellulose, carboxy methyl cellulose, poly itaconic acid, polyethyleneimine, ionomers, and/or natural polymers, such as gelatin, casein, xanthan gum, guar gum, gum arabic, carrageenan, pectin, etc. A variety of polymers can be used as the water-soluble layer 116, each one with a unique elongation at break, and their thickness can be modified to enhance the feel and/or improve the transfer film's 110 physical properties.

In some embodiments, the water-soluble layer 116 may have a thickness of about 1-10 μm. For example, the thickness of the water-soluble layer 116 may be between about 1-8 μm, 1-5 μm, 1-4 μm, 1-3 μm, 1-2 μm, 2-10 μm, 2-8 μm, 2-5 μm, 2-4 μm, 2-3 μm, 3-10 μm, 3- 8 μm, 3-6 μm, 3-5 μm, 3-4 μm, 4-10 μm, 4-8 μm, 4-5 μm, 5-10 μm, 5-8 μm, 5-6 μm, 6-10 μm, 6-8 μm, or 8-10 μm. The thickness of the water-soluble layer 116 may be greater than or equal to about 1, 2, 3, 4, 5, 6, 7, 8, or 9 μm. The thickness of the water-soluble layer 116 may be less than or equal to about 10, 9, 8, 7, 6, 5, 4, 3, or 2 μm.

The protective polymer layer 114 can provide protection for the cosmetic inkjet ink from abrasion and/or UV light, thereby impacting the longevity of the temporary tattoo. The protective polymer layer 114 may affect the finish (e.g., matte, glossy, etc.) of the temporary tattoo. The protective polymer layer 114 may comprise one or more clear (i.e., transparent) polymers, such as acrylic, polyurethane, polyethylene, polycarbonate, polyphenylene ether, polyvinylchloride, polypropylene, silicone elastomer, fluorinated ethylene propylene, styrene methyl methacrylate, polyethylene terephthalate glycol, cyclic olefin copolymers, styrene acrylonitrile, polystyrene, and/or acrylonitrile butadiene styrene (ABS).

The protective polymer layer 114 may have a thickness between about 1-10 microns. For example, the thickness of the protective polymer layer 114 may be between about 1-8 μm, 1-5 μm, 1-4 μm, 1-3 μm, 1-2 μm, 2-10 μm, 2-8 μm, 2-5 μm, 2-4 μm, 2-3 μm, 3-10 μm, 3-8 μm, 3-6 μm, 3-5 μm, 3-4 μm, 4-10 μm, 4-8 μm, 4-5 μm, 5-10 μm, 5-8 μm, 5-6 μm, 6-10 μm, 6-8 μm, or 8-10 μm. The thickness of the protective polymer layer 114 may be greater than or equal to about 1, 2, 3, 4, 5, 6, 7, 8, or 9 μm. The thickness of the protective polymer layer 114 may be less than or equal to about 10, 9, 8, 7, 6, 5, 4, 3, or 2 μm.

The inkjet receptive coating 112 may have a thickness between about 1-10 microns. For example, the thickness of the inkjet receptive coating 112 may be between about 1-8 μm, 1-5 μm, 1-4 μm, 1-3 μm, 1-2 μm, 2-10 μm, 2-8 μm, 2-5 μm, 2-4 μm, 2-3 μm, 3-10 μm, 3-8 μm, 3-6 μm, 3-5 μm, 3-4 μm, 4-10 μm, 4-8 μm, 4-5 μm, 5-10 μm, 5-8 μm, 5-6 μm, 6-10 μm, 6-8 μm, or 8-10 μm. The thickness of the inkjet receptive coating 112 may be greater than or equal to about 1, 2, 3, 4, 5, 6, 7, 8, or 9 μm. The thickness of the inkjet receptive coating 112 may be less than or equal to about 10, 9, 8, 7, 6, 5, 4, 3, or 2 μm.

The inkjet receptive coating 112 may be configured to absorb the cosmetic inkjet ink described herein. In some embodiments, the thickness of the inkjet receptive coating 112 may be greater than or equal to that of the cosmetic inkjet ink 102 to allow the ink to fully absorb into the coating. For example, the inkjet receptive coating 112 may be 10%, 15%, 20%, 25%, 30%, 35%, 40%, 45%, 50% or more greater in thickness than the cosmetic inkjet ink 102.

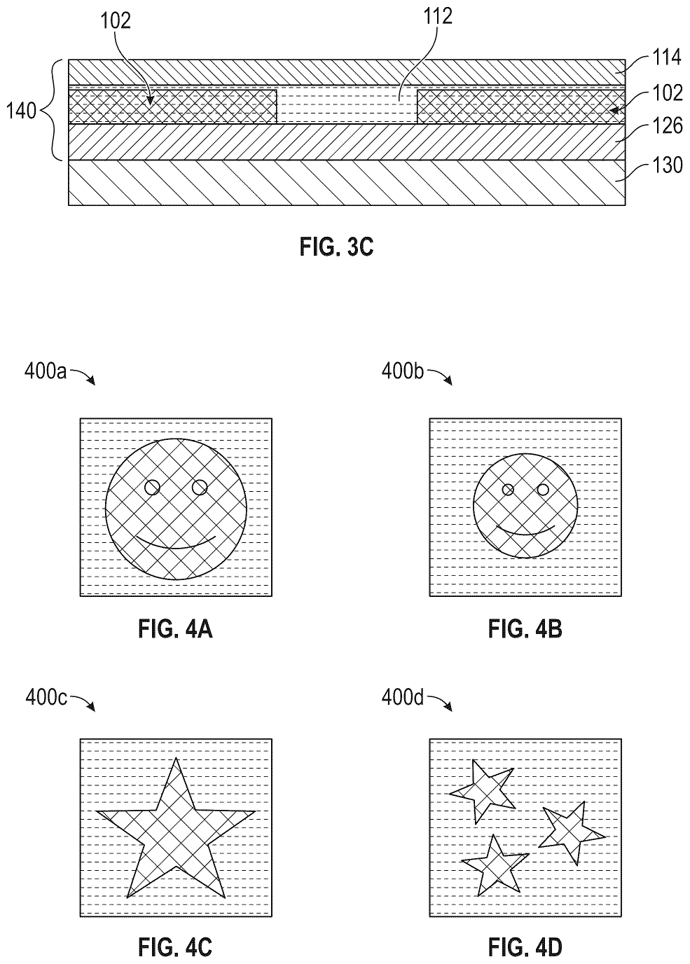

In some embodiments, only a portion of the inkjet receptive coating 112 may receive cosmetic inkjet ink 102 during printing, demonstrated by the void between the two inks 102 in the cross-sectional image in FIG. 1. As described herein, detailed images can be printed on the transfer film 110 (i.e., in the inkjet receptive coating 112 of the transfer film 110) using the cosmetic inkjet ink 102, which can leave some portions of the inkjet receptive coating 112 with ink, and some portions of the inkjet receptive coating 112 without ink. FIGS. 4A-4D illustrate exemplary temporary tattoos 400a, 400b, 400c, 400d with varying ratios of ink 102 to open areas on inkjet receptive coating 112. For example, the inkjet receptive coating 112 may surround a perimeter of a single inkjet image, as shown in FIGS. 4A-4C. In some examples, the printed inkjet image may have one or more open portions within the image itself, as shown in FIG. 4D.

In some embodiments, the printed inkjet image may comprise 10%, 20%, 25%, 30%, 40%, 50%, 60%, 70%, 75%, 80%, 90%, 95% or more of the inkjet receptive coating 112.

In some embodiments, the image printed in the cosmetic ink layer may have a resolution between about 360-2440 horizontal or vertical dots per inch (dpi). Printhead resolution is typically stated as two values: vertical dpi X horizontal dpi. Horizontal dpi can be determined by the frequency of drop firing and the carriage speed. Vertical dpi can be determined by the native resolution of the printhead or its nozzles per inch. The effective resolution can be determined by the native resolution and the number of greyscales it can achieve (drop sizes).

In each of the below example values and ranges of values for resolution, it is to be understood that the range of values can be the horizontal and/or the vertical dpi to provide the effective resolution. For example, the resolution of the printed image may be between about 500-2500 dpi, 600-2400 dpi, 700-2300 dpi, 800-2200 dpi, 900-2100 dpi, 1000-2000 dpi, 1100-1900 dpi, 1200-1800 dpi, 1300-1700 dpi, 1400-1600 dpi, 400-2000 dpi, 400-1800 dpi, 400-1600 dpi, 400-1400 dpi, 400-1200 dpi, 400-1000 dpi, 400-900 dpi, 400-800 dpi, 800-2400 dpi, 800-2200 dpi, 800-2000 dpi, 800-1800 dpi, 800-1600 dpi, 800-1400 dpi, 800-1200 dpi, 1000-2500 dpi, 1000-2200 dpi, 1000-1800 dpi, 1000-1500 dpi, 1000-1400 dpi, 1000-1200 dpi, 1200-2500 dpi, 1200-2200 dpi, 1200-2000 dpi, 1200-1600 dpi, 1200-1400 dpi, 1500-2500 dpi, 1500-1200 dpi, 1500-1000 dpi, 1500-1800 dpi, 1800-2500 dpi, 1800-2200 dpi, 2000-2500 dpi, or 2000-2200 dpi. In some embodiments, the resolution of the printed image in the cosmetic ink layer make be greater than or equal to about 360 dpi, 400 dpi, 450 dpi, 500 dpi, 550 dpi, 600 dpi, 650 dpi, 700 dpi, 750 dpi, 800 dpi, 850 dpi, 900 dpi, 950 dpi, 1000 dpi, 1050 dpi, 1100 dpi, 1150 dpi, 1200 dpi, 1250 dpi, 1300 dpi, 1350 dpi, 1400 dpi, 1450 dpi, or 1500 dpi. In some embodiments, the resolution of the printed image in the cosmetic ink layer may be less than or equal to about 2440 dpi, 2400 dpi, 2350 dpi, 2300 dpi, 2250 dpi, 2200 dpi, 2150 dpi, 2100 dpi, 2050 dpi, 2000 dpi, 1950 dpi, 1900 dpi, 1850 dpi, 1800 dpi, 1750 dpi, 1700 dpi, 1650 dpi, 1600 dpi, 1550 dpi, or 1500 dpi.

In some embodiments, the transfer film 110 can be modified (e.g., by modifying one or more of the existing layers and/or adding a layer) to cause the resulting end product to be either matte or glossy. For example, one or more additives can be added to one or more layers of the transfer film 110 and/or added to a new layer of the transfer film 110.

In some embodiments, one or more of the aforementioned layers of the transfer film 110 may be omitted or otherwise modified. For example, a toner-based temporary tattoo backing may not require an inkjet receptive coating 110 but may nonetheless comprise a water-soluble layer 116 on a side of the toner-based backing, a protective polymer layer 114 on a side of the water-soluble layer 116 opposite the backing, and a cosmetic ink layer comprising the cosmetic inkjet ink 102 on a side of the protective polymer layer 114 opposite the water-soluble layer 116.

As mentioned above, the temporary tattoo 100 may comprise an adhesive film 120. The adhesive film 120 may be used to ensure that the image created in the inkjet receptive layer 112 with cosmetic inkjet ink 102 can adhere to the skin once the backing 118 is removed. The adhesive film 120 can also prevent the cosmetic ink 102 from dissolving (e.g., with water) before use. The adhesive film 120 may comprise a backing 122, a wax layer 124 on a side of the backing 122, and an adhesive layer 126 on a side of the wax layer 124 opposite the backing 122. The backing 122 may comprise polyethylene terephthalate (PET). Before use of the temporary tattoo 100, the adhesive layer 126 may be on a side of the cosmetic ink layer 112 of the transfer film 110 opposite the protective polymer film 114, as shown in FIG. 1.

The adhesive layer 126 may comprise an acrylic material with a thickness between about 15-40 μm. For example, the thickness of the adhesive layer 126 may be between about 15-35 μm, 15-30 μm, 15-25 μm, 15-20 μm, 20-40 μm, 20-35 μm, 20-30 μm, 20-25 μm, 22-32 μm, 22-30 μm, 22-28 μm, 22-26 μm, 24-34 μm, 24-32 μm, 24-30 μm, 24-28 μm, 24-26 μm, 25-40 μm, 25-35 μm, or 25-30 μm. In some embodiments, the adhesive layer 126 may comprise a thickness greater than or equal to about 15, 20, 22, 24, 25, 26, 28, 30, 32, 34, or 35 μm. In some embodiments, the adhesive layer 126 may comprise a thickness less than or equal to about 40, 35, 34, 32, 30, 28, 26, 25, 24, 22, or 20 μm.

### Cosmetic Inkjet Ink Compositions

The cosmetic inkjet ink described herein may be composed of ingredients approved for use in cosmetic applications. The ingredients may include humectants, polymers, surfactants, additives, colorants, and/or solvents. For example, the cosmetic inkjet ink may comprise 10-25 wt. % of one or more humectants, 1-15 wt. % of one or more polymers, 0.01-2 wt. % of one or more surfactants, 0.01-5 wt. % of one or more additives, 2-20 wt. % of one or more colorants, and 60-80 wt. % of one or more solvents. Each of the ingredients of the formulation are described in greater detail below.

In some embodiments, the cosmetic inkjet ink formulation described herein may comprise one or more humectants. Humectants can improve the jetting of the ink using a piezo-based printhead and/or prevent the ink from prematurely drying. The cosmetic inkjet ink formulation may comprise 1-25 wt. % of one or more humectants. For example, the cosmetic inkjet ink formulation may comprise 1-22 wt. %, 1-20 wt. %, 1-18 wt. %, 1-15 wt. %, 1-12 wt. %, 1-10 wt. %, 1-8 wt. %, 1-5 wt. %, 5-25 wt. %, 5-22 wt. %, 5-20 wt. %, 5-18 wt. %, 5-15 wt. %, 5-12 wt. %, 5-10 wt. %, 5-8 wt. %, 10-25 wt. %, 10-22 wt. %, 10-20 wt. %, 12-25 wt. %, 12-20 wt. %, 14-25 wt. %, 14-22 wt. %, 14-20 wt. %, 16-25 wt. %, 16-22 wt. %, 16-20 wt. %, 18-25 wt. %, 18-22 wt. %, or 18-20 wt. % of one or more humectants. The cosmetic inkjet ink formulation may comprise greater than or equal to 1, 5, 10, 12, 14, 16, 18, 20, or 22 wt. % of one or more humectants. The cosmetic inkjet ink formulation may comprise less than or equal to 25, 22, 20, 18, 15, 12, 10, or 5 wt. % of one or more humectants.

The humectant of the cosmetic inkjet ink formulation may comprise one or more polyhydroxy compounds. For example, the cosmetic inkjet ink formulation may comprise 1,2-propanediol, 1,3-propanediol diglycerol, glycerol, glycerin, isoprene glycol, dipropylene glycol, Polyethylene glycol, Polypropylene glycol, Polybutylene glycol, glycol ethers, Dipropylene glycol monomethyl ether, Dipropylene glycol dimethyl ether, Dipropylene glycol n propyl ether, tripropylene glycol monomethyl ether, butoxytriglycol, and/or triethylene glycol monobutyl ether. In some embodiments, the humectant component of the cosmetic inkjet ink formulation may comprise glycerin, dipropylene glycol, and triethylene glycol monobutyl ether. In some embodiments, the humectant component of the cosmetic inkjet ink formulation may comprise glycerin, dipropylene glycol, and butoxytriglycol.

In some embodiments, the cosmetic inkjet ink formulation described herein may comprise one or more polymers. Polymers can be used to improve adhesion of the cosmetic inkjet ink to the transfer film and can aid in reducing the drying time of ink on the transfer film. Polymers added to the formulation can either be soluble in the solvent used or can be dispersed. The polymer can be naturally occurring or synthetic-based. The cosmetic inkjet ink formulation may comprise 1-25 wt. % of one or more polymers. For example, the cosmetic inkjet ink formulation may comprise 1-22 wt. %, 1-20 wt. %, 1-18 wt. %, 1-15 wt. %, 1-12 wt. %, 1-10 wt. %, 1-8 wt. %, 1-5 wt. %, 2-5 wt. %, 4-8 wt. %, 5-25 wt. %, 5-22 wt. %, 5-20 wt. %, 5-18 wt. %, 5-15 wt. %, 5-12 wt. %, 5-10 wt. %, 5-8 wt. %, 10-25 wt. %, 10-22 wt. %, 10-20 wt. %, 12-25 wt. %, 12-20 wt. %, 14-25 wt. %, 14-22 wt. %, 14-20 wt. %, 16-25 wt. %, 16-22 wt. %, 16-20 wt. %, 18-25 wt. %, 18-22 wt. %, or 18-20 wt. % of one or more polymers. The cosmetic inkjet ink formulation may comprise greater than or equal to 1, 2, 4, 5, 10, 12, 14, 16, 18, 20, or 22 wt. % of one or more polymers. The cosmetic inkjet ink formulation may comprise less than or equal to 25, 22, 20, 18, 15, 12, 10, or 5 wt. % of one or more polymers.

The polymer of the cosmetic inkjet ink formulation may comprise one or more of Polyethylene glycol (PEG), Polyvinylpyrrolidone, Polyvinyl caprolactone, Polylactones, Polyacrylic acid, Polyvinyl alcohol, Polyvinyl acrylamides, Polyamines, Carboxymethylcellulose, Hydroxyethylcellulose, Methylcellulose, Gelatin, Casein, Xanthan gum, Cellulose-based polymers and their derivatives, Guar gum, Gum Arabic, Carrageenan, and/or pectin. In some embodiments, the cosmetic inkjet ink formulation may comprise polyvinylpyrrolidone (PVP) and/or polyethylene glycol (PEG). For example, the cosmetic inkjet ink formulation may comprise PVP K-15. In some examples, the cosmetic inkket formulation comprises a cellulose-based polymer. In some examples, the cellulose-based polymer is Ambergum™ 3021.

In some embodiments, the cosmetic inkjet ink formulation described herein may comprise one or more surfactants. Surfactants can be used to improve the wetting and/or penetrability of the cosmetic inkjet ink on the transfer film. The cosmetic inkjet ink formulation may comprise 0.01-5 wt. % of one or more surfactants. For example, the cosmetic inkjet ink formulation may comprise 0.01-4 wt. %, 0.01-3 wt. %, 0.01-2 wt. %, 0.01-1 wt. %, 0.01-0.5 wt. %, 0.01-0.2 wt. %, 0.01-0.1 wt. %, 0.01-0.05 wt. %, 0.05-5 wt. %, 0.05-4 wt. %, 0.05-3 wt. %, 0.05-2 wt. %, 0.05-1 wt. %, 0.05-0.5 wt. %, 0.05-0.2 wt. %, 0.05-0.1 wt. %, 0.1-5 wt. %, 0.1-4 wt. %, 0.1-3 wt. %, 0.1-2 wt. %, 0.1-1 wt. %, 0.1-0.5 wt. %, 0.1-0.2 wt. %, 0.2-5 wt. %, 0.2-4 wt. %, 0.2-3 wt. %, 0.2-2 wt. %, 0.2-1 wt. %, 0.2-0.5 wt. %, 0.5-5 wt. %, 0.5-4 wt. %, 0.5-3 wt. %, 0.5-2 wt. %, 0.5-1 wt. %, 1-5 wt. %, 1-4 wt. %, 1-3 wt. %, or 1-2 wt. % of one or more surfactants. The cosmetic inkjet ink formulation may comprise greater than or equal to 0.01, 0.05, 0.1, 0.2, 0.5, or 1 wt. % of one or more surfactants. The cosmetic inkjet ink formulation may comprise less than or equal to 5, 4, 3, 2, 1, 0.5, 0.2, or 0.1 wt. % of one or more surfactants.

The surfactant of the cosmetic inkjet ink formulation may be anionic, cationic, zwitterionic, or non-ionic. Example surfactants that may be included in the cosmetic inkjet ink formulation described herein may include Sodium lauryl sulfate, Ammonium laureth sulfate, Disodium lauryl sulfosuccinate, Cocamphocarboxyglycinate, Cocoamidopropyl betaine, Alpha-Olefin sulfonate, Sodium cocoyl isethionate, Cetrimonium chloride, Sodium lauraminopropionate, Cocoamphopropionate, Ether sulfanates, Fatty glycerols, Monoglyceride sulfates, Propyl peptide condensates, Acyl sarcocinates, Acyl methyl taurates, Quaternary ammonium salts, Cetrimonium bromide, cetylpyrdinium chloride, octyl glucoside, fatty alcohols, polyoxyethylene sorbitan monolaurate, polyoxyethylene sorbitan monostearate, and/or glyceryl laurate. In some embodiments, the cosmetic inkjet ink may comprise Merpol® SH and/or Merpol® A.

In some embodiments, the cosmetic inkjet ink formulation described herein may comprise one or more additives. Additives may include one or more pH buffers, biocides, antioxidants, UV absorbers, chelating agents, etc. For example, the cosmetic inkjet ink formulation may comprise 0.01-5 wt. % of one or more pH buffers. pH buffers can be used to adjust the pH of the formulation and ensure that the pH range does not deviate. In some embodiments, the cosmetic inkjet ink formulation may comprise 0.01-4 wt. %, 0.01-3 wt. %, 0.01-2 wt. %, 0.01-1 wt. %, 0.01-0.5 wt. %, 0.01-0.2 wt. %, 0.01-0.1 wt. %, 0.01-0.05 wt. %, 0.05-5 wt. %, 0.05-4 wt. %, 0.05-3 wt. %, 0.05-2 wt. %, 0.05-1 wt. %, 0.05-0.5 wt. %, 0.05-0.2 wt. %, 0.05-0.1 wt. %, 0.1-5 wt. %, 0.1-4 wt. %, 0.1-3 wt. %, 0.1-2 wt. %, 0.1-1 wt. %, 0.1-0.5 wt. %, 0.1-0.2 wt. %, 0.2-5 wt. %, 0.2-4 wt. %, 0.2-3 wt. %, 0.2-2 wt. %, 0.2-1 wt. %, 0.2-0.5 wt. %, 0.5-5 wt. %, 0.5-4 wt. %, 0.5-3 wt. %, 0.5-2 wt. %, 0.5-1 wt. %, 1-5 wt. %, 1-4 wt. %, 1-3 wt. %, or 1-2 wt. % of one or more pH buffers. The cosmetic inkjet ink formulation may comprise greater than or equal to 0.01, 0.05, 0.1, 0.2, 0.5, or 1 wt. % of one or more pH buffers. The cosmetic inkjet ink formulation may comprise less than or equal to 5, 4, 3, 2, 1, 0.5, 0.2, or 0.1 wt. % of one or more pH buffers.

The pH buffer of the cosmetic inkjet ink formulation described herein may comprise one or more amines, such as dimethylaminopropylamine, Triethanol amine, and/or aminomethyl propanediol. In some embodiments, the pH buffer may comprise sodium hydroxide and/or ammonium hydroxide.

In some embodiments, the cosmetic inkjet ink formulation may comprise 0.1-5 wt. % of one or more biocides. In some embodiments, the cosmetic inkjet ink formulation may comprise 0.1-5 wt. %, 0.1-4 wt. %, 0.1-3 wt. %, 0.1-2 wt. %, 0.1-1 wt. %, 0.1-0.5 wt. %, 0.1-0.2 wt. %, 0.2-5 wt. %, 0.2-4 wt. %, 0.2-3 wt. %, 0.2-2 wt. %, 0.2-1 wt. %, 0.2-0.5 wt. %, 0.5-5 wt. %, 0.5-4 wt. %, 0.5-3 wt. %, 0.5-2 wt. %, 0.5-1 wt. %, 0.8-5 wt. %, 0.8-4 wt. %, 0.8-3 wt. %, 0.8-2 wt. %, 0.8-1 wt. % 1-5 wt. %, 1-4 wt. %, 1-3 wt. %, 1-2 wt. %, 2-5 wt. %, 2-4 wt. %, or 2-3 wt. % of one or more biocides. The cosmetic inkjet ink formulation may comprise greater than or equal to 0.1, 0.2, 0.5, 0.8, 1, or 2 wt. % of one or more biocides. The cosmetic inkjet ink formulation may comprise less than or equal to 5, 4, 3, 2, 1, 0.5, or 0.2, wt. % of one or more biocides.

The biocide of the cosmetic inkjet ink formulation may comprise one or more isothiazolinones, parabens, organic acids, formaldehyde donors, botanical oils, and/or phenoxyethanols. For example, the cosmetic inkjet ink formulation may comprise one or more of benzothiazolinone (BIT), Methylisothiazolinone (MIT), methyl paraben, ethyl paraben, butyl paraben, propyl paraben, benzoic acid, Dimethylol-5,5-dimethylhydantoin, Imidazolidinyl urea, Diazolidinyl urea, Quaternium 15, Benzyl alcohol, Rosemary oil extract, Neem oil, Lavender oil, Tea tree oil, Halogenated preservatives, bronopol, phenoxyethanol, and/or ethylhexylglycerin. In some examples, the biocide comprises Optiphen™, a known biocide consisting of phenoxyethanol and an emollient base.

In some embodiments, the cosmetic inkjet ink formulation described herein may comprise one or more colorants. The colorant of the cosmetic inkjet ink formulation may comprise one or more cosmetic approved dyes and/or cosmetic approved pigments. The pigments are typically dyes that have been precipitated out using metallic salts and are otherwise commonly referred to as lakes. This process makes the organic dyes insoluble in water. Dyes that can be used in temporary tattoos can be artificially or naturally obtained. The cosmetic dyes may be more vibrant than the typical dye lakes or inorganic pigments used in temporary tattoo inks. In some embodiments, the cosmetic inkjet ink formulation may comprise 1-25 wt. % of one or more colorants. For example, the cosmetic inkjet ink formulation may comprise 1-22 wt. %, 1-20 wt. %, 1-18 wt. %, 1-15 wt. %, 1-12 wt. %, 1-10 wt. %, 1-8 wt. %, 1-5 wt. %, 2-25 wt. %, 2-22 wt. %, 2-20 wt. %, 2-18 wt. %, 2-15 wt. %, 2-12 wt. %, 2-10 wt. %, 2-8 wt. %, 2-5 wt. %, 5-25 wt. %, 5-22 wt. %, 5-20 wt. %, 5-18 wt. %, 5-15 wt. %, 5-12 wt. %, 5-10 wt. %, or 5-8 wt. % of one or more colorants. The cosmetic inkjet ink formulation may comprise greater than or equal to 1, 2, or 5 wt. % of one or more colorants. The cosmetic inkjet ink formulation may comprise less than or equal to 25, 22, 20, 18, 15, 12, 10, or 5 wt. % of one or more colorants.

The colorant of the cosmetic inkjet ink formulation may comprise one or more naturally occurring, synthetic, organic, or inorganic pigments and/or dyes. For example, the cosmetic inkjet ink formulation may comprise one or more of Red Iron Oxide (Red and Blue Shade), Russet Iron Oxide, Brown Iron Oxide, Yellow Iron Oxide, Chromium Oxide Green, Hydrated Chromium Oxide Green, Iron Blue, Ultramarine Blue, Ultramarine Pink, Ultramarine Violet, Manganese Violet, Black Iron Oxide, Titanium Dioxide.

In some embodiments, the cosmetic inkjet ink formulation may comprise one or more of FD&C Red 4, Red 14, Red 17, Red 22, Red 28, Red 33, Red 40, Red 504, Red 227, Orange 4, Yellow 4, Yellow 5, Yellow 6, Yellow 7, Yellow 8, Yellow 10, Quinoline Yellow, Green 3, Green 5, Green 6, Green 8, Blue 1, Blue 5, Violet 2, D&C Red 6 Ba Lake, D&C Red 7 Ca Lake, D&C Red 27 Al Lake, D&C Red 28 Al Lake, D&C Red30 Al Lake, FD&C Blue 1 Al Lake, FD&C Yellow 5 Al Lake. In some examples, the cosmetic inkjet ink formulation comprises a combination of Red 33 and Red 22. In some examples, the cosmetic inkjet ink formulation comprises Red 33. In some examples, the cosmetic inkjet ink formulation comprises Red 22.

Naturally occurring dyes that may be included in the cosmetic inkjet ink formulations described herein are provided below in Table 1, reproduced from U.S. Patent Application No. 2012/0141398, the entire contents of which are incorporated herein by reference.

TABLE 1
Naturally occurring dyes
List of Natural Food Colors

| | | Main |
|---|---|---|
| Colors | Plant | Composition |
| Plum Colors | Plum | Anthocyanins |
| Purple Corn Colors | Maiz morado | Anthocyanins |
| Red Cabbage Colors | Brassica oleracea L. var. | Anthocyanins |
| | Capitata DC. ev. Red Acre | |
| Blueberry Color | Vaccinium corymbosum L. | Anthocyanins |
| Cherry Colors | Prunus pauciflora BUNCH | Anthocyanins |
| Grape Juice Colors | Vitis vinifera L. | Anthocyanins |
| Grape Skin Colors | Red grapes | Anthocyanins |
| Hibiscus Colors | Hibiscus sabdariffa L. | Anthocyanins |
| Mulberry Colors | Morus nigra L., M. alba L. | Anthocyanins |
| Perilla Colors | Perilla | Anthocyanins |
| Strawberry Colors | Fragaria ananassa | Anthocyanins |
| | DUCHESNE | Anthocyanins |
| Aronia Berries Colors | Prunus virginiana | Anthocyanins |
| Blackcurrant Fruit Colors | Ribes nigrum | Anthocyanins |
| Black Carrot Colors | Daucus carota | Anthocyanins |
| Elderberries Colors | Elderberries | Anthocyanins |
| Gardenia Blue | Gardenia | Genipin |
| Gardenia Yellow | Gardenia augusta MERR. | Crocin |
| | vargracliflora HORT | |
| Annatto, water or | Bixa orellana L. | Norbixin; |
| oil soluble | | Bixin |
| Amaranthus Colors | Red amaranth | Amaranthin |
| Cocoa Colors | Theobro macacao | Flavonoids |
| Licorice Colors | Glycyrrhiza glabra L., | Flavonoids |
| | Glycyrrhiza uralensis FISCH. | |
| Buckwheat Extract | Fagopyrum esculentum | Flavonoids |
| | MOENCH | |
| Saffron | Crocus sativus L. | Flavonoids |
| Safflower Yellow | Carthamus tinctorius | Flavonoids |
| Onion Colors | Allium cepa L. | Flavonoids |
| Peanut Colors | Arachis hypogaea L. | Flavonoids |
| Sorghum Colors | Sorghum | Flavonoids |
| Caramel | Sugar | Sugar |
| Sugar Cane Colors | Sugar | Sugar |
| Malted Barley Colors | Malted barley | Sugar |
| Turmeric | Curcuma longa | Curcumin |
| Carmine | Coccus cacti L. | Carminic Acid |
| Laver Colors | Porphyra tenera KJELLM. | Phycoerythrin |
| Spirulina Colors | Spirulina | Phycocyanin |
| Monascus Colors | Monascus purpureus, | |
| | Monascusanka | |
| Beet Red | Beta vulgaris | Betanin |
| Tamarind Color | Tamarindus indica L. | Polyphenol |
| Persimmon Colors | Persimmon | Polyphenol |
| Chlorella Colors | Chlorella | Chlorophyll |
| Chlorophyll Colors | Green plant | Chlorophyll |
| Stinging Nettle Colors | Urtica dioica | Chlorophyll |
| Spinach Colors | Spinacia oleracea | Chlorophyll |
| Carrot Colors | Daucus carota subsp. Sativus | β-Carotene |
| Sweet Potato Colors | Ipomoea batatas POIR. | β-Carotene |
| Corn Colors | Zea mays L. | Carotenoids |
| Crawfish Colors | Crab | Carotenoids |
| Orange Colors | Orange | Carotenoids |
| Paprika Colors | Capsicum annum | Carotenoids |
| Shrimp Colors | Shrimp | Carotenoids |
| Tomato Colors | Tomato | Lycopene |
| Aztec Marigold Colors | Aztec marigold | Xanthophylls |
| Xanthophylls | Alfalfa | Xanthophylls |
| SandalWood Colors | Pterocarpus santalinus) | Santalin |
| Sepiomelanin Colors | Sepia officinalis | Sepiomelanin |

Synthetic dyes and pigments that can be included in the cosmetic inkjet ink formulations described herein are provided in Table 2, reproduced from U.S. Patent Application No. 2012/0141398, the entire contents of which are incorporated herein by reference.

TABLE 2
Synthetic dyes and pigments
List of Food Synthetic Colors

| | | | | Names of | Names of |
|---|---|---|---|---|---|
| | | | | Food | Food |
| | | | | Colors | Colors |
| | | | | Used in | Used in |
| No. | CI No. | CAS No. | Colors | U.S.A. | Taiwan |
| 1 | 12156 | 6358-53-8 | Citrus Red | Citrus | |
| | | | No. 2 | Red No. 2 | |
| 2 | 15985 | 2783-94-0 | FD&C | FD&C | Taiwan Food |
| | | | Yellow | Yellow | Yellow No. 5 |
| | | | No. 6 | No. 6 | |
| 3 | 15985:1 | 15790-07-5 | FD&C | FD&C | Taiwan Food |
| | | | Yellow | Yellow | Yellow No. 5 |
| | | | No. 6 Lakes | No. 6 Lakes | |
| 4 | 16035 | 25956-17-6 | FD&C Red | FD&C Red | Taiwan Food |
| | | | No. 40 | No. 40 | Red No. 40 |
| 5 | 16035:1 | 68583-95-9 | FD&C Red | FD&C Red | Taiwan Food |
| | | | No. 40 Lakes | No. 40 Lakes | Red No. 40 |
| 6 | 16255 | 2611-82-7 | Acid Red 18 | | Taiwan Food |
| | | | | | Red No. 6 |
| 7 | 19140 | 1934-21-0 | FD&C | FD&C | Taiwan Food |
| | | | Yellow | Yellow | Yellow No. 4 |
| | | | No. 5 | No. 5 | |
| 8 | 19140:1 | 12225-21-7 | FD&C | FD&C | Taiwan Food |
| | | | Yellow | Yellow | Yellow No. 4 |
| | | | No. 5 Lakes | No. 5 Lakes | |
| 9 | 19235 | 15139-76-1 | Orange B | Orange B | |
| 10 | 42053 | 2353-45-9 | FD&C Green | FD&C Green | Taiwan Food |
| | | | No. 3 | No. 3 | Green No. 3 |
| 11 | 42090 | 3844-45-9 | FD&C Blue | FD&C Blue | Taiwan Food |
| | | | No. 1 | No. 1 | Blue No. 1 |
| 12 | 42090:2 | 68921-42-6 | FD&C Blue | FD&C Blue | Taiwan Food |
| | | | No. 1 Lakes | No. 1 Lakes | Blue No. 1 |
| 13 | 45430 | 16423-68-0 | FD&C Red | FD&C Red | Taiwan Food |
| | | | No. 3 | No. 3 | Red No. 7 |
| 14 | 45430:1 | 12227-78-0 | FD&C Red | FD&C Red | Taiwan Food |
| | | | No. 3 Lakes | No. 3 Lakes | Red No. 7 |
| 15 | 73015 | 860-22-0 | FD&C Blue | FD&C Blue | Taiwan Food |
| | | | No. 2 | No. 2 | Blue No. 2 |
| 16 | 73015:1 | 16521-38-3 | FD&C Blue | FD&C Blue | Taiwan Food |
| | | | No. 2 | No. 2 | Blue No. 2 |
| | | | Aluminum | Aluminum | |
| | | | Lake | Lake | |

Cosmetic dyes and pigments that can be included in the cosmetic inkjet ink compositions described herein are provided below in Tables 3A-D, reproduced from U.S. Patent Application No. 2012/0141398, the entire contents of which are incorporated herein by reference.

TABLE 3
Cosmetic colors
List of Cosmetic Colors

| No. | CI No. | CAS No. | Colors |
|---|---|---|---|
| 1 | 10020 | 19381-50-1 | Ext. D&C Green No. 1 |
| 2 | 10316 | 846-70-8 | Ext. D&C Yellow No. 7. |
| 3 | 11380 | 85-84-7 | Ext. D&C Yellow No. 9 |
| 4 | 11390 | 131-79-3 | Ext. D&C Yellow No. 10 |
| 5 | 11680 | 2512-29-0 | Ext. D&C Yellow No. 5 |
| 6 | 11710 | 6486-23-3 | Pigment Yellow 3 |
| 7 | 11725 | 6371-96-6 | Ext. D and C Orange No. 1 |
| 8 | 11743 | 12236-75-8 | Pigment Yellow 105 |
| 9 | 12085 | 2814-77-9 | D&C Red No. 36. |
| 10 | 12010 | 6535-42-8 | Solvent Red 3 |
| 11 | 12100 | 2646-17-5 | Ext. D&C Orange No. 4 |
| 12 | 12120 | 2425-85-6 | D&C Red No. 35 |
| 13 | 12140 | 3118-97-6 | Ext. D&C Red No. 14 |
| 14 | 12150 | 1229-55-6 | Solvent Red 1 |
| 15 | 12156 | 6358-53-8 | Citrus Red No. 2 |
| 16 | 12315 | 6448-95-9 | Pigment Red 22 |
| 17 | 12490 | 6410-41-9 | Pigment Red 5 |
| 18 | 13015 | 2706-28-7 | Acid Yellow 9 |
| 19 | 13058 | 6371-55-7 | D&C Red No. 39. |
| 20 | 13065 | 587-98-4 | Ext. D&C Yellow No. 1 |
| 21 | 14600 | 523-44-4 | Ext. D&C Orange No. 3 |
| 22 | 14700 | 4548-53-2 | FD&C Red No. 4 |
| 23 | 14720 | 3567-69-9 | EXT D & C Red No. 10 |
| 24 | 15510 | 633-96-5 | D&C Orange No. 4. |
| 25 | 15585:1 | 5160-02-1 | D&C Red No. 9 |
| 26 | 15620 | 1658-56-6 | Ext. D&C Red No. 8 |
| 27 | 15630 | 1248-18-6 | D&C Red No. 10 |
| 28 | 15630:1 | 1103-38-4 | D&C Red No. 12 |
| 29 | 15630:2 | 1103-39-5 | D&C Red No. 11 |
| 30 | 15630:3 | 6371-67-1 | D&C Red No. 13 |
| 31 | 15800 | 6371-76-2 | D&C Red No. 31. |
| 32 | 15850 | 5858-81-1 | D&C Red No. 6. |
| 33 | 15850:1 | 5281-04-9 | D&C Red No. 7 |
| 34 | 15850:2 | 17852-98-1 | D&C Red No. 6, barium lake |
| 35 | 15865 | 3564-21-4 | Pigment Red 48 |
| 36 | 15880 | 21416-46-6 | D&C Red No. 34. |
| 37 | 15880:1 | 6417-83-0 | D&C Red No. 34 |
| 38 | 15985 | 2783-94-0 | FD&C Yellow No. 6 |
| 39 | 15985:1 | 15790-07-5 | FD&C Yellow No. 6 Lakes |
| 40 | 16035 | 25956-17-6 | FD&C Red No. 40 |
| 41 | 16035:1 | 68583-95-9 | FD&C Red No. 40 Lakes |
| 42 | 16150 | 3761-53-3 | D&C Red No. 5 |
| 43 | 16155 | 3564-09-8 | FD&C Red No. 1 |
| 44 | 16185 | 915-67-3 | FD&C Red No. 2 |
| 45 | 16185:1 | 12227-62-2 | FD&C Red No. 2 Lakes |
| 46 | 16255 | 2611-82-7 | Acid Red 18 |
| 47 | 16255:1 | 12227-64-4 | Pigment Red 274 |
| 48 | 17200 | 3567-66-6 | D&C Red No. 33. |
| 49 | 18050 | 3734-67-6 | Ext D&C Red No. 11 |
| 50 | 18820 | 6359-82-6 | Ext. D&C Yellow No. 3 |
| 51 | 18950 | 6372-96-9 | Ext. D&C Yellow No. 4 |
| 52 | 19140 | 1934-21-0 | FD&C Yellow No. 5 |
| 53 | 19140:1 | 12225-21-7 | FD&C Yellow No. 5 Lakes |
| 54 | 19235 | 15139-76-1 | Orange B |
| 55 | 20170 | 1320-07-6 | D&C Brown No. 1. |
| 56 | 20470 | 1064-48-8 | D&C Black No. 1. |
| 57 | 21090 | 6358-85-6 | Pigment Yellow 12 |
| 58 | 21110 | 3520-72-7 | Pigment Orange 13 |
| 59 | 26100 | 85-86-9 | D&C Red No. 17. |
| 60 | 26105 | 85-83-6 | Solvent Red 24 |
| 61 | 42052 | 3374-30-9 | D&C Blue No. 7 |
| 62 | 42053 | 2353-45-9 | FD&C Green No. 3 |
| 63 | 42085 | 4680-78-8 | FD&C Green No. 1 |
| 64 | 42090 | 3844-45-9 | FD&C Blue No. 1 |
| 65 | 42090 | 2650-18-2 | D&C Blue No. 4 |
| 66 | 42090:1 | 6548-12-5 | Pigment Blue 24 |
| 67 | 42090:2 | 68921-42-6 | FD&C Blue No. 1 Lakes |
| 68 | 42095 | 5141-20-8 | D&C Green No. 4 |
| 69 | 42640 | 1694-09-3 | FD&C Violet No. 1 |
| 70 | 45100 | 3520-42-1 | Acid Red 52 |
| 71 | 45160:1 | 12224-98-5 | Pigment Red 81 |
| 72 | 45170:3 | 12227-77-9 | Pigment Red 173 |
| 73 | 45190 | 6252-76-2 | Ext. D&C Red No3 |
| 74 | 45350 | 518-47-8 | D&C Yellow No. 8. |
| 75 | 45350:1 | 2321-07-5 | D&C Yellow No. 7. |
| 76 | 45370:1 | 596-03-2 | D&C Orange No. 5. |
| 77 | 45370:2 | 93776-20-6 | Pigment Orange 39 |
| 78 | 45380 | 17372-87-1 | D&C Red No. 22. |
| 79 | 45380:2 | 15086-94-9 | D&C Red No. 21. |
| 80 | 45380:3 | 15876-39-8 | Pigment Red 90:1 |
| 81 | 45410 | 18472-87-2 | D&C Red No. 28. |
| 82 | 45410:1 | 2134-15-8 | D&C Red No. 27. |
| 83 | 45410:2 | 15876-58-1 | Pigment Red 174 |
| 84 | 45425 | 33239-19-9 | D&C Orange No. 11. |
| 85 | 45425:1 | 38577-97-8 | D&C Orange No. 10. |
| 86 | 45430 | 16423-68-0 | FD&C Red No. 3 |
| 87 | 45430:1 | 12227-78-0 | FD&C Red No. 3 Lakes |
| 88 | 45440 | 11121-48-5 | Acid Red 94 |
| 89 | 47000 | 8003-22-3 | D&C Yellow No. 11. |
| 90 | 47005 | 8004-92-0 | D&C Yellow No. 10. |
| 91 | 47005:1 | 68814-04-0 | D&C Yellow No. 10. Lakes |
| 92 | 51319 | 6358-30-1 | Pigment Violet 23 |
| 93 | 58000:1 | 104074-25-1 | Pigment Red 83 |
| 94 | 59040 | 6358-69-6 | D&C Green No. 8. |
| 95 | 60725 | 81-48-1 | D&C Violet No. 2. |
| 96 | 60730 | 4430-18-6 | Ext. D&C Violet No. 2. |
| 97 | 61520 | 6408-50-0 | Solvent Blue 63 |
| 98 | 61565 | 128-80-3 | D&C Green No. 6. |
| 99 | 61570 | 4403-90-1 | D&C Green No. 5. |
| 100 | 69825 | 130-20-1 | D&C Blue No. 9. |
| 101 | 73000 | 482-89-3 | D&C Blue No. 6. |
| 102 | 73015 | 860-22-0 | FD&C Blue No. 2 |
| 103 | 73015:1 | 16521-38-3 | FD&C Blue No. 2 Aluminum Lake |
| 104 | 73360 | 2379-74-0 | D&C Red No. 30. |
| 105 | 74160 | 147-14-8 | Pigment Blue 15 |
| 106 | 74260 | 1328-53-6 | Pigment Green 7 |
| 107 | 74350 | 1328-54-7 | Solvent Blue 25 |
| 108 | 75170 | 73-40-5 | Natural White 1 |
| 109 | 77019 | 12001-26-2 | Mica-based pearlescent colors |
| 110 | 77266 | 1333-86-4 | D&C Black No. 2 |

The cosmetic inkjet ink formulation described herein may comprise one or more solvents. Solvent in the cosmetic inkjet ink may function as a carrier fluid in the formulation. In some embodiments, the cosmetic inkjet ink formulation may comprise 50-90 wt. % of one or more solvents. For example, the cosmetic inkjet ink formulation may comprise 50-85 wt. %, 50-80 wt. %, 50-75 wt. %, 50-70 wt. %, 55-90 wt. %, 55-85 wt. %, 55-80 wt. %, 55-75 wt. %, 55-70 wt. %, 55-65 wt. %, 60-90 wt. %, 60-85 wt. %, 60-80 wt. %, 60-75 wt. %, 60-70 wt. %, 65-90 wt. %, 65-85 wt. %, 65-80 wt. %, 65-75 wt. %, 65-70 wt. %, 70-90 wt. %, 70-85 wt. %, 70-80 wt. %, or 70-75 wt. % of one or more solvents. The cosmetic inkjet ink formulation may comprise greater than or equal to 50, 55, 60, 65, or 70 wt. % of one or more solvents. The cosmetic inkjet ink formulation may comprise less than or equal to 90, 85, 80, 75, 70, or 65 wt. % of one or more solvents.

The solvent of the cosmetic inkjet ink formulation may comprise one or more glycol ethers, diols, esters, and/or water. For example, the cosmetic inkjet ink formulation may comprise ethanol and/or water. The water can be deionized water.

Each of the ingredients in the cosmetic inkjet ink formulations described herein can be selected to ensure proper jetting performance using inkjet printing processes and to meet cosmetic standards. All ingredients (including colorants) for the cosmetic inkjet ink described herein may not contain components listed on the restricted cosmetic ingredients lists from the US (eCFR Title 21, Chapter I, Subchapter G, Part 700, Subpart B) and EU (Annex II of Regulation (EC) No. 1223/2009) regulatory bodies. The cosmetic inkjet ink described herein can be printed directly to a film and/or directly to the skin.

### Methods of Making Temporary Tattoos Using Cosmetic Inkjet Ink

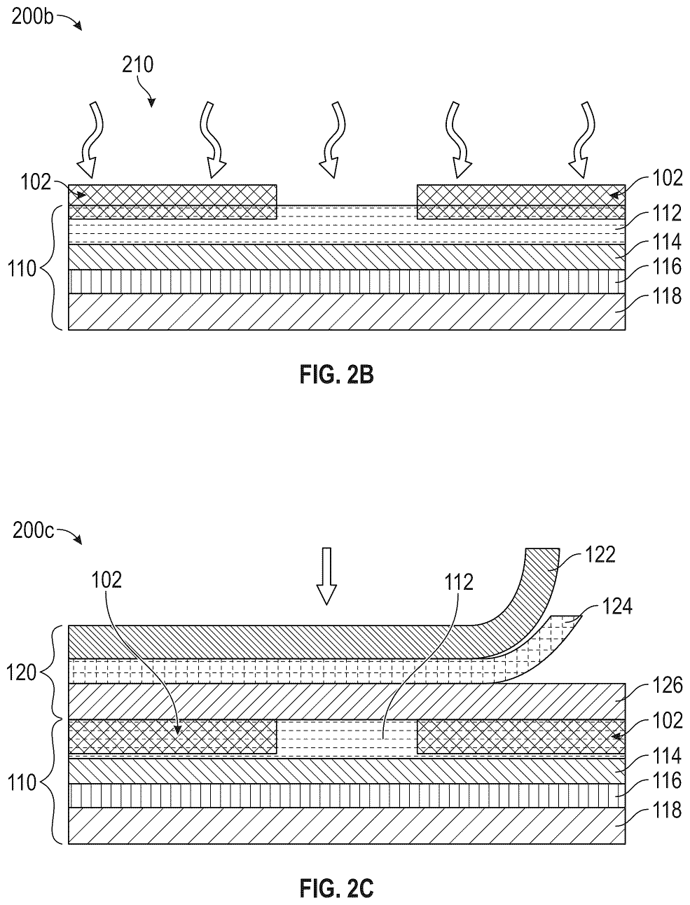

FIGS. 2A-2C illustrate various steps of a method for making a temporary tattoo (e.g., temporary tattoo 100) using cosmetic inkjet ink.

FIG. 2A shows step 200a in producing a temporary tattoo. Step 200a can include printing, using an inkjet printer 205, an image made from a cosmetic inkjet ink 102 on a side of a transfer film 110 comprising an inkjet receptive coating 112. As shown in FIG. 2A, when the ink 102 is initially printed to the inkjet receptive coating 112 of the transfer film 110, the ink may not initially be fully received by the coating 112, and instead may require a period of time to dry and be fully absorbed into the receptive coating 112.

In some embodiments, the inkjet printer may comprise piezo printheads or thermal printheads. However, it is to be understood that any printer than can print inkjet ink can be reasonably used with the printing methods described herein. The printer may or may not contain heat zones to dry the film. The use of heating to dry the film may be dependent on the film that is used. For example, whether heat is used to dry the film may be dependent on the thickness of the ink receptive layer of the film.

Suitable inkjet printers can include but are not limited to: Epson SureColor F170, Epson SureColor F570, Epson SureColor F6470/H, Epson Surecolor F7200, Epson SureColor F10070/H, Epson SureColor F9470/H, Epson SureColor R5070, Roland Texart RT-640/M, Roland Texart XT-640, Mutoh ValueJet 2638WX, Mutoh ValueJet 1948WX, Mutoh ValueJet 1938WX, Mutoh XpertJet 1682WR, Mutoh XpertJet 1642 WR Pro, Mutoh R-900x, Hewlett Packard Stitch S300, Hewlett Packard Stitch S500, Hewlett Packard 2700, Hewlett Packard Latex 700/800, Hewlett Packard 300, Canon DGI Poseidon Dye Sublimation Printer, Canon DGI FH-3204, Canon DGI FT-3204X, Canon Colorado 1650, Canon Colorado 1630, Mimaki TS55-1800, Mimaki TS100-1600, Mimaki TS300P-1800, Mimaki TS330-1600, Mimaki TS500-1800, Mimaki TS500-3200, Mimaki Tiger-1800B MkIII, Mimaki CJV150, Miamki CJV300Plus, Miamki TX300P-1800B, Mimaki TX500P-3200DS, and Mimaki JV400LX Series.

FIG. 2B shows step 200b in producing a temporary tattoo. Step 200b can include drying the image printed on the transfer film 110 such that the image (i.e., the inkjet ink 102) absorbs into the inkjet receptive coating 112. In some embodiments, drying the image may comprise air-drying and/or using a heat source 210 to dry the image on the transfer film 110. As mentioned above, in some embodiments the inkjet printer may comprise a heat source for drying the image. In some embodiments, drying the ink 102 of the image may include placing and/or hovering the coated film comprising the image underneath or on top of a heat source 210 until the image adequately dries. The heat source may include conductive heating or an infrared radiative heater. If using a heat source 210 to dry the image on the transfer film 110, a temperature setting of the heat source 210 may be between about 20-50° C. Drying may occur in-line during the printing process, or alternatively, the transfer film 110 can be cut and drying can occur off-line. In some embodiments, the composition of the cosmetic inkjet ink 102 may be formulated to ensure that it can dry at ambient temperature. For example, the use of polyvinylpyrrolidone in one or more layers of the transfer film 110 may contribute to the process of air-drying the image printed to the transfer film 110. Any heating process can be used, so long as the heat does not degrade layers of the transfer film.

FIG. 2C shows step 200c in producing a temporary tattoo. Step 200c can include applying an adhesive film 120 to the side of the transfer film 110 comprising the inkjet receptive coating 112 to cover the image. As shown, applying the adhesive film 120 to the side of the transfer film 110 comprising the inkjet receptive coating 112 may comprise applying the adhesive layer 126 of the adhesive film 120 to the inkjet receptive coating 112 of the transfer film 110.

### Methods of Using Temporary Tattoos Made Using Cosmetic Inkjet Ink

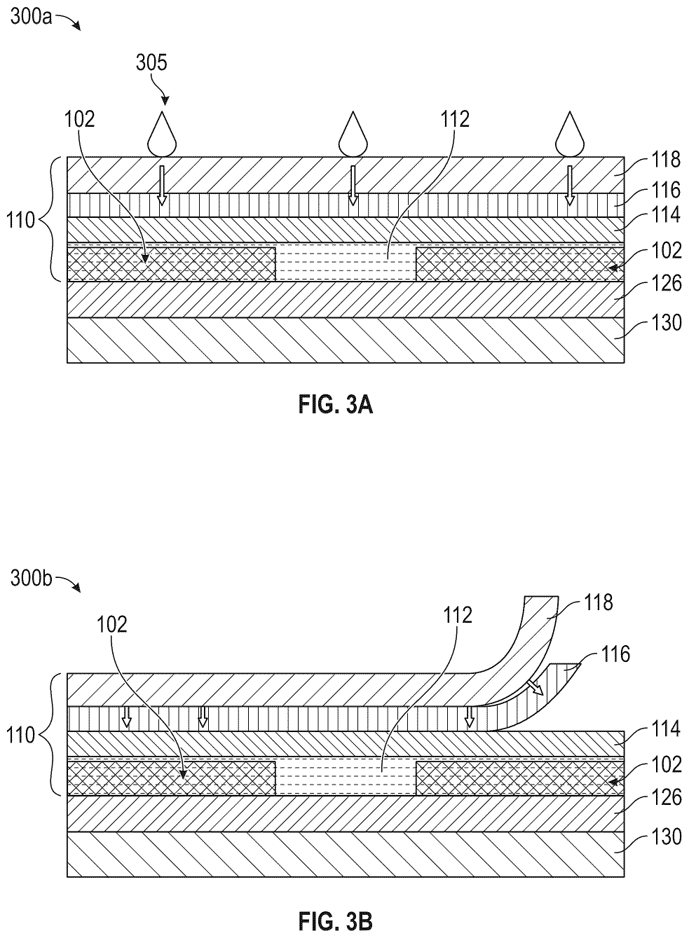

FIGS. 3A-3C illustrate various steps of a method for using a temporary tattoo (e.g., temporary tattoo 100) that have been produced using cosmetic inkjet ink. Using the temporary tattoo may otherwise be referred to herein as applying the temporary tattoo to skin.

FIG. 3A shows step 300a in applying the temporary tattoo to the body. Prior to applying the tattoo, the adhesive film backing 122 can be removed, for example by peeling the backing 122 (and the wax layer 124) from the adhesive layer 126. Once the backing 122 is removed, step 300a can include applying the adhesive layer 126 to a desired area of the body 130. Once adhered, water 305 can be applied to the print backing 118, which can saturate the backing 118 to reach the water-soluble layer 116 and dissolve the layer 116. For example, water 305 can be applied by applying a wet (e.g., damp) towel to the backing 118 for a variable amount of time.

FIG. 3B shows step 300b in applying the temporary tattoo to the body. Step 200b can include removing the saturated print backing 118 (and the dissolved water-soluble layer 116, which is understood to be a unit with the backing 118). Removing the print backing 118 can include peeling the backing 118 away from the body 130. Once the print backing 118 is removed, the printed inkjet image within the inkjet receptive coating 112 is left behind with a protective polymer layer 114 on a side of the inkjet receptive coating 112 opposite the body 130.

FIG. 3C shows a cross-sectional view of the final temporary tattoo product adhered to the body 130 and protected by a protective polymer layer 114. The final tattoo on the skin may comprise an adhesive layer 126 on the skin, a cosmetic ink layer on a side of the adhesive 126 opposite the body 130 and comprising an inkjet receptive coating 112 and an image printed using cosmetic inkjet ink 102, and a protective polymer layer 114 on a side of the cosmetic ink layer opposite the adhesive layer 126. As shown in FIG. 3C and described herein, the image printed using the cosmetic inkjet ink 102 may result in portions of the inkjet receptive coating 112 with ink, and other portions of the coating without ink. Whether the inkjet receptive coating 112 is with or without ink throughout different portions of the tattoo may have no effect on the performance of the adhesive layer 126 in adhering the tattoo to the skin and the protective polymer layer 114 in protecting the tattoo from abrasion, water, and UV light.

### Testing

As is evident from the above disclosure, the temporary tattoos provided herein can be composed from a variety of different materials with varying properties. For example, different materials can be used in the various layers of the transfer film described herein. Moreover, different thicknesses of the layers of the film can be used herein. Material selection and amount of material can directly affect the performance (e.g., water proofing, durability, longevity, re-solubility, etc.) of the tattoo. A unique combination of these materials (and the amounts of these materials) may be determined as described herein to provide a temporary tattoo with optimized performance and characteristics.

To determine temporary tattoo compositions that exhibit the desired performance and characteristics, experimentation can be performed. Exemplary testing protocols for determining characteristics of the tattoo, including water fastness, removal from skin, adhesive residue, gloss level, and re-solubility are described below. Exemplary methods for preparing the temporary tattoos and applying the prepared tattoos for testing are also provided below.

Preparation: A cosmetic inkjet ink can be printed directly onto a temporary tattoo transfer film comprising an inkjet receptive coating on a side of the transfer film. The ink may be designed to ensure minimal heat was required to dry (i.e., absorb) the image printed to the inkjet receptive layer. The cosmetic inkjet ink can comprise humectants, surfactants, additives (e.g., biocides, antioxidants, UV absorbers, chelating agents, etc.) and a cosmetic grade dye or pigment. The cosmetic inkjet ink can be printed using a piezo or thermal head printer. Once the image is printed and dried, an adhesive film can be applied to the side of the transfer film comprising the inkjet receptive coating.

Application: To use the temporary tattoo, the backing of the adhesive film can be removed, and the remaining adhesive layer can be applied to the skin. Water can then be applied by either spraying, running under a stream, or using a wet cloth or paper towel to soak the paper-based print backing on the external surface of the temporary tattoo. Once the backing is saturated, it can be removed from the surface by gradually sliding it off to expose the printed image. The remaining external layer, the protective polymer layer, can serve as a waterproof and abrasion resistance layer that ensures the ink of the printed image does not run.

Water fastness: To test water fastness, a wet cloth or paper towel can be applied to the temporary tattoo on the skin (i.e., the wet towel is in contact with the above-described protective polymer). The cloth/paper towel can be visually observed to determine whether any ink has transferred onto the towel. The results can be qualitatively classified as follows: ink observed on the towel may be considered “poor” and no ink observed on the towel may be considered “good.” The temporary tattoos provided in accordance with the formulations and compositions described herein may demonstrate good water fastness.

Removal from skin: To test the removal of the temporary tattoo from the skin, once the temporary tattoo is transferred to the skin, the tattoo can be peeled off the skin. How the tattoo is affected during the process of removal can be visually observed. The results ca be qualitatively classified based on whether the tattoo removes in one piece or several pieces (i.e., “breaks apart”). The temporary tattoos provided in accordance with the formulations and compositions described herein may remove in one piece.

Adhesive residue: To test whether, after removing the temporary tattoo from the skin, any adhesive is left on the skin, the area on the skin can be touched and qualitatively classified for tackiness. The results can be classified as follows: adhesive still present may be considered “significant” and no adhesive present (i.e., the adhesive was removed along with the remaining portions of the tattoo) may be considered “none.” The temporary tattoos provided in accordance with the formulations and compositions described herein may leave no adhesive residue on the skin.

Gloss Level: To test the gloss level of the temporary tattoo, the standard operating procedure ASTM D523 can be used to measuring the gloss level of the temporary tattoos on different substrates, including leather and aluminum. The criteria presented in the procedure indicate a 60° angle for measurements. Five measurements (in units) for each tattoo being tested in different locations of the tattoo can be conducted, and an average of the measurements can be determined. The temporary tattoos provided in accordance with the formulations and compositions described herein may have a gloss level on leather between 10-25 units, such as between about 10-20, 10-15, 15-25, or 15-20 units. The temporary tattoos provided in accordance with the formulations and compositions described herein may have a gloss level on aluminum between 30-60 units, such as between about 30-50, 30-40, 40-60, or 50-60 units.

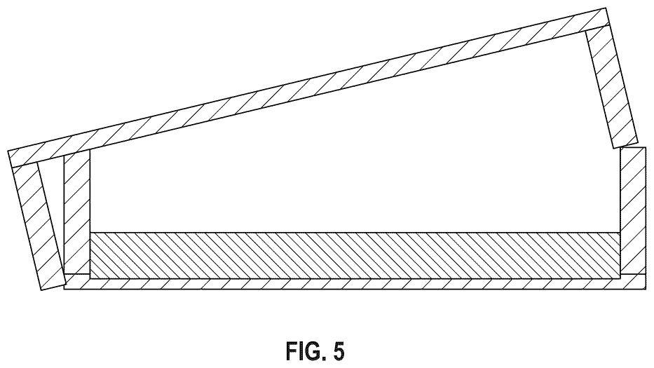

Re-solubility: To test re-solubility of temporary tattoo formulations, solutions of dye and/or polymer can be created. The solution can include deionized water as solvent. An amount (e.g., about 20 grams) of the solution can be placed in a dish with walls (e.g., a petri dish) and a cover can be placed on top of the dish at an angle, as shown in FIG. 5, to allow for the evaporation of water and any other volatiles. The dish can be placed in an oven at a set temperature and for a set amount of time to dry, such as at about 60° C. and for about 24 hours. Once dry, the dish can be removed from the oven to cool. About 20 grams of deionized water can be added back to the dish to re-solubilize the precipitate. The solution can be mixed using a spatula to agitate the precipitate for about 1 minute. The ease of incorporation of the precipitate into the water can be noted. The solution can be left to rest for about a minute, and after the minute has passed, the ease of incorporation of the precipitate into the water can again be tested. The solution can be assigned a score representative of the ease of incorporation of the precipitate back into solution. For example, a grading scale between, e.g., 1-5, 1-10, 1-3, etc. can be used to assess the ease of incorporation of the precipitate into the solution, with 1 being easy to incorporate, and the end value (e.g., 3, 5, 10) being not re-dissolving at all. An example grading scale is provided below with respect to Example 4. The temporary tattoos provided in accordance with the formulations and compositions described herein may have a re-solubility score of 1 or 2, meaning the solutions are very soluble or semi-soluble with slight or little to no agitation.

### EXAMPLES

The examples provided below are not intended to limit the scope of the disclosure provided herein.

### Example 1: Temporary Tattoo Composition

An example temporary tattoo formulation with relevant thicknesses of layers of the tattoo is provided in Table E1 below.

TABLE E1
Example Temporary Tattoo Composition

| | |
|---|---|
| Layer | Thickness (μm) |

Transfer Film

| | |
|---|---|
| Print backing | 192 |
| Water soluble layer | 5 |
| Protective polymer layer- | 1-2 |
| polyurethane | |
| Protective polymer layer- | 3 |
| acrylic | |
| Inkjet receptive coating | 3 |

Adhesive Film

| | |
|---|---|
| Acrylic adhesive | 25 |
| Silicone/wax | — |
| Adhesive backing-PET | — |

### Example 2: Cosmetic Inkjet Ink Formulations

Example cosmetic inkjet ink formulations for use in temporary tattoos are provided below in Tables E2, E3, and E4. Tables E2 and E3 include cosmetic inkjet ink formulations for cyan, magenta, yellow, and black, indicated accordingly. Tables E2 and E3 also provide properties of each of the formulations, including viscosity (measured at 20° C.), pH, and surface tension (referred to as S/T in the tables).

TABLE E2
Example Cosmetic Inkjet Ink Formulation A

| | Cyan 1 | Magenta 1 | Yellow 1 | Black 1 |
|---|---|---|---|---|
| Deionized Water | 69.89 | 71.88 | 69.89 | 69.85 |
| Glycerine | 7.20 | 7.00 | 7.20 | 7.20 |
| Dipropylene Glycol | 4.60 | 2.00 | 4.60 | 4.60 |
| Butoxytriglycol | 7.20 | 6.00 | 7.20 | 7.20 |
| Euxyl PE9010 | 0.90 | 0.90 | 0.90 | 0.90 |
| Aminomethyl | 0.20 | 0.20 | 0.20 | 0.20 |
| Propanediol | | | | |
| Merpol ® A | 0.01 | 0.02 | 0.01 | 0.05 |
| PVP K-15 | 6.00 | 8.00 | 6.00 | 6.00 |
| Unicert Blue 1 | 4.00 | | | 1.55 |
| Unicert Red 28 | | 4.00 | | 1.55 |
| Unicert Yellow 5 | | | 4.00 | 0.90 |
| TOTAL | 100.00 | 100.00 | 100.00 | 100.00 |
| Viscosity 20C (cP) | 4.58 | 4.48 | 4.56 | 4.57 |
| S/T (mN/m) | 30.83 | 31.52 | 30.61 | 29.5 |
| pH | 8.35 | 8.35 | 8.35 | 8.35 |

Formulation B provided in Table E3 differs from Formulation A provided in Table E2 because the amount of polymer (PVP K-15) was reduced, a different biocide was used, and different dyes were used. Reducing the amount of PVP K-15 improved the solubility of the formulation. Reducing the PVP K-15 was preferable to changing the type of polymer to, for example, a cellulose-based polymer because the glass transition with cellulose-based polymer was lower and the formulation with cellulose-based polymer was tackier. Changing the type of polymer to polyacrylic acid also resulted in a lower glass transition and tackier formulation. Because the amount of polymer was reduced, the amount of humectant in Formulation B was also increased to compensate for the decrease and maintain the viscosity of the formulation.

Formulation B also uses different magenta and black dyes as compared to Formulation A. Magenta in Formulation B uses a combination of FDC Red 33 and FDC Red 22, whereas magenta in Formulation A uses Red 28. It was found that FDC Red 33 does not penetrate into the lower plastic adhesive layer of the temporary tattoo. Since black dye is a combination of cyan, magenta, and yellow, the dyes for black were changed between Formulation A and Formulation B, too, to include a combination of FDC Red 33 and FDC Red 22.

Finally, in Formulation B the biocide used was Optiphen™ rather than Euxyl PE9010 as used in Formulation A. Optiphen™ was found to be more compatible with the cosmetic inkjet ink of Formulation B as compared to Euxyl PE9010.

TABLE E3
Example Cosmetic Inkjet Ink Formulation B

| | Cyan | Magenta | Yellow | Black |
|---|---|---|---|---|
| Deionized Water | 66.78 | 69.68 | 69.79 | 66.79 |
| Glycerine | 8.00 | 8.00 | 7.20 | 8.00 |
| Dipropylene Glycol | 8.00 | 8.00 | 4.60 | 8.00 |
| Butoxytriglycol | 8.00 | 8.00 | 7.20 | 8.00 |
| Optiphen ™ | 1.00 | 1.00 | 1.00 | 1.00 |
| Aminomethyl Propanediol | 0.20 | 0.20 | 0.20 | 0.20 |
| Merpol ® A | 0.02 | 0.02 | 0.01 | 0.02 |
| PVP K-15 | 4.00 | 4.00 | 6.00 | 4.00 |
| Unicert Blue 05601-J No. 1 | 4.00 | | | 1.33 |
| Unicert Red K7057-J No. 33 | | 0.70 | | 0.93 |
| Unicert Red K7008-J No. 22 | | 0.4 | | 0.399 |
| Unicert Yellow 08005-J No. 5 | | | 4.00 | 1.33 |
| TOTAL | 100.00 | 100.00 | 100.00 | 100.00 |
| Viscosity 20C (cP) | 4.01 | 3.95 | 4.30 | 4.15 |
| S/T (mN/m) | 30.64 | 32.48 | 28.93 | 31.35 |
| pH | 9.06 | 8.94 | 9.33 | 9.18 |

Table E4 provides an example cosmetic inkjet ink formulation C that includes blue dye. It is to be understood that any color dye (e.g., magenta, yellow, black, etc.) described herein may reasonably be substituted to the composition.

TABLE E4
Example Cosmetic Inkjet Ink Formulation C

| Ingredient | Wt. % |
|---|---|
| Glycerin | 7.2 |
| Dipropylene Glycol | 4.6 |
| Triethylene Glycol | 7.2 |
| Monobutyl Ether | |
| PVP K-15 | 6 |
| Merpol ® A | .01 |
| Aminomethyl | .2 |
| propanediol | |
| Phenoxyethanol and | .9 |
| Ethylhexylglycerin | |
| FD&C Blue No. 1 Dye | 4 |
| (other dyes available) | |
| Water | 69.89 |

Each of the ingredients in the cosmetic inkjet ink formulations described herein have been selected to ensure proper jetting performance using inkjet printing processes and to meet cosmetic standards. All ingredients (including colorants) for the cosmetic inkjet ink described herein do not contain components listed on the restricted cosmetic ingredients lists from the US (eCFR Title 21, Chapter I, Subchapter G, Part 700, Subpart B) and EU (Annex II of Regulation (EC) No. 1223/2009) regulatory bodies. The cosmetic inkjet ink described herein can be printed directly to a film and/or directly to the skin.

### Example 3: Testing Water Fastness, Removal from Skin, Adhesive Residue, and Gloss Level of Various Temporary Tattoo Compositions

To determine temporary tattoo compositions that exhibit the desired performance and characteristics, experimentation was performed. Testing protocols for determining characteristics of the tattoo, including water fastness, removal from skin, adhesive residue, and gloss level are described below. Example methods for preparing the temporary tattoos and applying the prepared tattoos for testing are also provided below.

### Methods

Testing samples: Five temporary tattoos comprising different protective polymer layers (i.e., waterproof films) of varying thicknesses were created. The samples were as follows: (1) 13-micron (μm) polyurethane, (2) 6-micron polyurethane, (3) 5-micron polyacrylate, (4) 3-micron polyacrylate, and (5) a combination of 3-micron polyurethane and 3-micron polyacrylate.

Preparation: For each of the above-described samples, a cosmetic inkjet ink was printed directly onto a temporary tattoo transfer film comprising an inkjet receptive coating on a side of the transfer film. The ink was designed to ensure minimal heat was required to dry (i.e., absorb) the image printed to the inkjet receptive layer. The cosmetic inkjet ink included humectants, surfactants, additives (e.g., biocides, antioxidants, UV absorbers, chelating agents, etc.) and a cosmetic grade dye or pigment. The cosmetic inkjet ink was printed using a piezo or thermal head printer. Once the image was printed and dried, an adhesive film was applied to the side of the transfer film comprising the inkjet receptive coating.

Application: To use the temporary tattoo, the backing of the adhesive film was removed, and the remaining adhesive layer was applied to the skin. Water was then applied by either spraying, running under a stream, or using a wet cloth or paper towel to soak the paper-based print backing on the external surface of the temporary tattoo. Once the backing was saturated, it was removed from the surface by gradually sliding it off to expose the printed image. The remaining external layer, the protective polymer layer, serves as a waterproof and abrasion resistance layer that ensures the ink of the printed image does not run.

Water fastness: To test water fastness, a wet cloth or paper towel was applied to the temporary tattoo on the skin (i.e., the wet towel is in contact with the above-described protective polymer). The cloth/paper towel was visually observed to determine whether any ink has transferred onto the towel. The results were qualitatively classified as follows: ink observed on the towel was considered “poor” and no ink observed on the towel was considered “good.”

Removal from skin: To test the removal of the temporary tattoo from the skin, once the temporary tattoo was transferred to the skin, the tattoo was peeled off the skin. How the tattoo was affected during the process of removal was visually observed. The results were qualitatively classified based on whether the tattoo removed in one piece or several pieces (i.e., “broke apart”).

Adhesive residue: To test whether, after removing the temporary tattoo from the skin, any adhesive was left on the skin, the area on the skin was touched and qualitatively classified for tackiness. The results were classified as follows: adhesive still present was considered “significant” and no adhesive present (i.e., the adhesive was removed along with the remaining portions of the tattoo) was considered “none.”

Gloss Level: To test the gloss level of the temporary tattoo, the standard operating procedure ASTM D523 was used to measuring the gloss level of the temporary tattoos on different substrates, including leather and aluminum. The criteria presented in the procedure indicated a 60° angle for measurements. Five measurements (in units) for each tattoo being tested in different locations of the tattoo were conducted, and an average of the measurements was determined.

### Results

Table E5 provided below demonstrates testing results for the temporary tattoos with polyurethane and/or polyacrylate protective layers at different thicknesses.

TABLE E5
Protective polymer layer testing results

| | | | | Adhesive | | |
|---|---|---|---|---|---|---|
| | | | | Residue | Gloss | Gloss |
| | | | Removal | Left | Level on | Level on |
| Water Proof | Thickness | Water | from | Behind on | Leather | Aluminum |
| Film | (μm) | Fastness | Skin | Skin | (60°) | (60°) |
| Thick | 13-micron | Good | Removes | None | 19 | 54 |
| Polyurethane | | | in one | | | |
| | | | piece | | | |
| Thin | 6-micron | Good | Removes | None | 19 | 54 |
| Polyurethane | | | in one | | | |
| | | | piece | | | |
| Thick | 5-micron | Poor | Breaks | Significant | 5 | 13 |
| Polyacrylate | | | apart | | | |
| Thin | 3-micron | Poor | Breaks | Significant | 5 | 13 |
| Polyacrylate | | | apart | | | |
| Combination | 3-micron | Good | Removes | None | 11 | 32 |
| | Polyurethane/3- | | in one | | | |
| | micron | | piece | | | |
| | Polyacrylate | | | | | |

As shown in the Table E5, a protective polymer layer including at least polyurethane exhibited the desired characteristics. The thickness of the polyurethane layer did not have a significant effect on the tested characteristics of the tattoo (i.e., water fastness, removal from skin, adhesive residue, gloss level). A protective polymer layer including a combination of polyurethane and polyacrylate exhibited similar water fastness, removal from skin, and adhesive residue to a layer having substantially only polyurethane. However, the gloss level of the layers was different. The combination layer was more matte (i.e., less glossy) than the polyurethane layer. Each of the layers had an “eggshell-like” finish on leather. On aluminum, the polyurethane layer was “semi-glossy,” whereas the combination layer was “satin-like.”

### Example 4: Testing Re-solubility of Various Temporary Tattoo Formulations

To determine temporary tattoo formulations that exhibit the desired re-solubility of a temporary tattoo, experimentation was performed. A testing protocol for assessing re-solubility of temporary tattoo formulations was developed and is described below.

### Methods

Solutions of dye and polymer (or polymer/humectant) as solute and deionized water as solvent were prepared based on the amount of solute (e.g., dye and polymer) used in inkjet ink temporary tattoo formulations described herein (e.g., formulations A-C provided above). 20 grams (g) of the solution was placed in a petri dish and a cover was placed on top of the petri dish at an angle (as shown in FIG. 5) to allow for the evaporation of water and any other volatiles. The petri dish was placed in a 60° C. oven for 24 hours to dry. Once the solution in the petri dish was dry, it was removed from the oven and allowed to cool. Then 20 g of deionized water was added to the petri dish. A spatula was used to mix the precipitate into the water for 1 minute, noting the ease of incorporation of the precipitate into the water. The solution was left to sit for another minute without stirring and, after the minute, the ease of incorporation of the solute into the water can again be tested. the solution was assigned a score from 1 to 5 in accordance with the grading scale shown in Table E6 below, with 1 being easy to incorporate back into solution and 5 being not re-dissolving at all.
E6. Grading scale for assessing re-solubility of dye/polymer solutions

| Num- | | % Solids |
|---|---|---|
| ber | Description | Remaining |
| 1 | Very Soluble: able to resolubilize | 0-20 |
| | into a liquid solution; requires | |
| | little to no agitation | |
| 2 | Semi-Soluble: able to resolubilize, | 20-40 |
| | but some (less than half) of | |
| | solids remain; requires slight agitation | |
| 3 | Not Very Soluble: around half of | 40-60 |
| | the solids remain with high | |
| | agitation | |
| 4 | Not Soluble: More than half the | 60-80 |
| | solids remain even with high | |
| | agitation | |
| 5 | Not Soluble: Close to all the | 80-100 |
| | solids remain even with high | |
| | agitation | |

### Results

The solutions tested and their respective re-solubility scores are shown below in Tables E7 and E8. The solutions shown in Table E7 varied the polymer/humectant but kept constant the type of dye used. The solutions shown in Table E8 kept constant the polymer but varied the dye(s) used.

TABLE E7
Polymer/Humectant & D&C Red 28 Solutions

| Polymer/Humectant | | | | Re-solubility |
|---|---|---|---|---|
| (wt. %) | | Dye (wt. %) | | Score |
| Glycerine | 8 | D&C Red 28 | 4 | 1 |
| Polyacrylic acid | 8 | D&C Red 28 | 4 | 1 |
| Ambergum ™ 3021 | 8 | D&C Red 28 | 4 | 1 |
| Sorez 100 | 8 | D&C Red 28 | 4 | 3 |
| Lupasol F | 8 | D&C Red 28 | 4 | 5 |
| Polyethylene glycol | 8 | D&C Red 28 | 4 | 5 |
| Poval 5-88 | 8 | D&C Red 28 | 4 | 5 |

TABLE E8
Polyvinylpyrollidone (PVP) K-15 & Dye Solutions

| | | | | Re-solubility |
|---|---|---|---|---|
| Polymer (wt. %) | | Dye (wt. %) | | Score |
| PVP K-15 | 8 | D&C Red 28 | 4 | 4 |
| PVP K-15 | 8 | FD&C Blue 1 | 4 | 4 |
| PVP K-15 | 8 | FD&C Red 33 | 4 | 3 |
| PVP K-15 | 8 | FD&C Red 22 | 4 | 3 |
| PVP K-15 | 8 | FD&C Yellow 5 | 4 | 2 |
| PVP K-15 | 8 | FD&C Red 33 | 0.7 | 2 |
| | | FD&C Red 22 | 0.4 | |
| PVP K-15 | 8 | FD&C Blue 1 | 1.55 | 2 |
| | | D&C Red 28 | 0.9 | |
| | | FD&C Yellow 5 | 1.55 | |

As shown in Table E7, solutions including polymer/humectants such as glycerine, polyacrylic acid, or Ambergum 3021 in combination with D&C Red 28 dye were found to be very soluble. These solutions required little to no agitation to resolubilize. On the other hand, solutions including polymer/humectants such as Lupasol F, polyethylene glycol, or Poval 5-88 in combination with D&C Red 28 dye were not found to be soluble. Close to all solids remained in these solutions, even with high agitation.

As shown in Table E8, the dye used can affect the re-solubility of polymer. Based on the dyes that exhibited improved re-solubility (yellow 5, red 33, and red 22), it is theorized that dye characteristics such as lower molecular weight and reduced halogenation of the dye structure may contribute to improving re-solubility. The multicomponent dye solutions aid in showing that mixtures including those dyes will jet and perform well in the field.

### Definitions

Although the preceding description uses terms first, second, etc. to describe various elements, these elements should not be limited by the terms. These terms are only used to distinguish one element from another.

This application discloses several numerical ranges in the text and figures. The numerical ranges disclosed inherently support any range or value within the disclosed numerical ranges, including the endpoints, even though a precise range limitation is not stated verbatim in the specification because this disclosure can be practiced throughout the disclosed numerical ranges.

Also, it is also to be understood that the singular forms “a,” “an,” and “the” used in the preceding description are intended to include the plural forms as well unless the context indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

The term “if” may be construed to mean “when” or “upon” or “in response to determining” or “in response to detecting,” depending on the context.

### CONCLUSION

The preceding description sets forth exemplary methods, parameters, and the like. It should be recognized, however, that such description is not intended as a limitation on the scope of the present disclosure but is instead provided as a description of exemplary embodiments. The illustrative embodiments described above are not meant to be exhaustive or to limit the disclosure to the precise forms disclosed. Many modifications and variations are possible in view of the above teachings. The embodiments were chosen and described to best explain the principles of the disclosed techniques and their practical applications. Others skilled in the art are thereby enabled to best utilize the techniques, and various embodiments with various modifications as are suited to the particular use contemplated.

Although the disclosure and examples have been thoroughly described with reference to the accompanying figures, it is to be noted that various changes and modifications will become apparent to those skilled in the art. Such changes and modifications are to be understood as being included within the scope of the disclosure and examples as defined by the claims. In the preceding description of the disclosure and embodiments, reference is made to the accompanying drawings, in which are shown, by way of illustration, specific embodiments that can be practiced. It is to be understood that other embodiments and examples can be practiced, and changes can be made without departing from the scope of the present disclosure.

What is claimed is:

1. A method of making a temporary tattoo, comprising:
printing, using an inkjet printer, an image made from a cosmetic inkjet ink on a side of a transfer film comprising an inkjet receptive coating;
drying the image printed on the transfer film such that the image absorbs into the inkjet receptive coating; and
applying an adhesive film to the side of the transfer film comprising the inkjet receptive coating to cover the image.

2. The method of claim 1, wherein the printed image has a resolution of 360-2440 horizontal or vertical dots per inch (dpi).

3. The method of claim 1, wherein the inkjet receptive coating has a thickness between 1-10 microns.

4. The method of claim 1, wherein the transfer film comprises a print backing, a water-soluble layer on a side of the print backing, and a protective polymer layer on a side of the water-soluble layer opposite the print backing.

5. The method of claim 4, wherein the inkjet receptive coating is on a side of the protective polymer layer opposite the water-soluble layer.

6. The method of claim 4, wherein the protective polymer layer has a thickness of 1-10 microns.

7. The method of claim 1, wherein the adhesive film comprises a backing, a wax layer on a side of the backing, and an adhesive layer on a side of the wax layer opposite the backing.

8. The method of claim 7, wherein applying the adhesive film to the side of the transfer film comprising the inkjet receptive coating comprises applying the adhesive layer of the adhesive film to the inkjet receptive coating of the transfer film.

9. The method of claim 1, wherein the inkjet printer comprises piezo printheads or thermal printheads.

10. The method of claim 1, wherein drying the image comprises air-drying or using heat to dry the image on the transfer film.

11. A temporary tattoo having a cosmetic inkjet ink-printed image, comprising:
a print backing;
a water-soluble layer on a side of the print backing;
a protective polymer layer on a side of the water-soluble layer opposite the print backing;
a cosmetic ink layer on a side of the protective polymer layer opposite the water-soluble film, the cosmetic ink layer comprising:
an inkjet receptive coating; and
a cosmetic inkjet ink absorbed into the inkjet receptive coating; and
an adhesive layer on a side of the cosmetic ink layer opposite the protective polymer film.

12. The temporary tattoo of claim 11, wherein the cosmetic ink layer has a resolution of 360-2440 horizontal or vertical dpi.

13. The temporary tattoo of claim 11, wherein the inkjet receptive coating has a thickness of 1-10 microns.

14. The temporary tattoo of claim 11, wherein the protective polymer layer has a thickness of 1-10 microns.

15. The temporary tattoo of claim 11, wherein the cosmetic inkjet ink comprises 10-25 wt. % humectant.

16. The temporary tattoo of claim 15, wherein the humectant comprises one or more of diglycerol, glycerin isoprene glycol, dipropylene glycol, tripropylene glycol monomethyl ether, and triethylene glycol monobutyl ether.

17. The temporary tattoo of claim 11, wherein the cosmetic inkjet ink comprises 1-15 wt. % polymer.

18. The temporary tattoo of claim 17, wherein the polymer comprises polyethylene glycol or polyvinylpyrrolidone.

19. The temporary tattoo of claim 11, wherein the cosmetic inkjet ink comprises 0.01-2 wt. % surfactant.

20. The temporary tattoo of claim 11, wherein the cosmetic inkjet ink comprises 2-20 wt. % of one or more cosmetic approved dyes and/or cosmetic approved pigments.

21. The temporary tattoo of claim 11, wherein the cosmetic inkjet ink comprises 0.01-2 wt. % pH buffer.

22. The temporary tattoo of claim 11, wherein the pH buffer comprises dimethylaminopropylamine or aminomethyl propanediol.

23. The temporary tattoo of claim 11, wherein the cosmetic inkjet ink comprises 0.1-3 wt. % biocide.

24. The temporary tattoo of claim 23, wherein the biocide comprises one or more of benzothiazolinone, phenoxyethanol, and ethylhexylglycerin.

25. The temporary tattoo of claim 11, wherein the cosmetic inkjet ink comprises 60-80 wt. % solvent.

26. The temporary tattoo of claim 25, wherein the solvent comprises water or ethanol.

27. The temporary tattoo of claim 11, wherein the print backing is a porous paper-based substrate.

28. The temporary tattoo of claim 11, wherein the protective polymer layer comprises acrylic and/or polyurethane.

29. The temporary tattoo of claim 11, wherein the water-soluble layer comprises polyvinyl alcohol.

30. The temporary tattoo of claim 11, comprising an adhesive film, the adhesive film comprising a backing, a wax layer on a side of the backing, and the adhesive layer on a side of the wax layer opposite the backing.

31. The temporary tattoo of claim 30, wherein the backing comprises polyethylene terephthalate.
